(19) United States
(12) Patent Application Publication
(10) Pub. No.: US 2021/0358602 A1
(43) Pub. Date: Nov. 18, 2021

(54) Dynamic Telemedicine Temporary Staffing System and Method

(71) Applicant: John Pannucci, Bridgewater, NJ (US)

(72) Inventor: John Pannucci, Bridgewater, NJ (US)

(21) Appl. No.: 16/876,093

(22) Filed: May 17, 2020

(51) Int. Cl. G16H 40/20; G16H 40/67; G06Q 20/08; G16H 80/00; G16H 70/20; G16H 50/20; G06Q 10/10; G06Q 30/04; H04L 29/08; H04L 12/58

(52) U.S. Cl. CPC G16H 40/20; G16H 40/67; G06Q 20/085; G16H 80/00; G16H 70/20; H04L 51/04; G06Q 10/1053; G06Q 30/04; H04L 67/12; H04L 51/16; G16H 50/20

(57) ABSTRACT

A method and system for remote dynamic telemedicine temporary staffing are provided, comprising a remote healthcare provider Internet server operated by a remote healthcare provider, a first Internet enabled communication device operated by a participating client, a telemedicine temporary staffing provider Internet server operated by a telemedicine temporary staffing provider, a second Internet enabled communication device is operated by a participating medical provider, and a remote payment service Internet server operated by a remote payment service. Said system is configured to provide telemedicine temporary staffing services to a remote healthcare provider by a remote telemedicine temporary staffing provider, said services includes executing a remote diagnostic sessions. Said system also creates receipts memorializing said remote diagnostic session, payment requests to remote payment service for participating medical providers for said remote diagnostic sessions and invoices requesting payment to remote healthcare providers by remote telemedicine temporary staffing providers for said remote diagnostic sessions.

### CROSS-REFERENCE(S)

None.

### FIELD OF THE INVENTION

The present disclosure relates to a dynamic telemedicine temporary staffing service and systems used to provide licensed medical professionals to telemedicine providers on a real time basis, more particularly, to provide telemedicine providers with licensed medical professionals to provide services to patients of said telemedicine providers dynamically.

### BACKGROUND OF THE INVENTION

Telehealth enables care delivery that helps hospitals, health systems and group practices deliver quality care at a distance. It often results in gains of efficiencies, improve patient and provider satisfaction and save costs.

For patient health and safety, Telehealth reduces the need for face-to-face visits. By providing licensed medical professionals care quickly and virtually, this approach has the potential to increase patient satisfaction and improve outcomes Licensed medical professionals are licensed by an authorized agency such as a Board of Medicine or a state department of labor and industry. For example, anyone in any medical or medical related profession who requires a State license to practice such as doctors, dentists, podiatrists, nurses, nurse practitioners, physician assistants, physical/occupational therapists, pharmacists, EMTs and others.

As federal officials remove many of the regulatory and reimbursement barriers that have hindered more widespread telehealth adoption in the past, telehealth providers are using new technologies to deliver care including Internet accessible platforms to increase support rapid implementation capability, minimal workflow disruption and non-limited volume capacity. The platform enables these telemedicine providers to quickly and cost-effectively launch licensed medial professional tele-consultations using their own staff.

Consequently, a system of telehealth communications networks have sprung into existence. Said telehealth communications networks accessible may be implemented anywhere sufficient bandwidth connection is available so as to facilitates a remote medical meeting between a patient at a patient site and a provider at a provider site remotely located from said patient.

Said telehealth communications networks require staffing by medical doctors and other licensed professionals to operated properly and lawfully. Due to the training and licensing requirements associated licensed professionals proper healthcare provider staffing regularly requires temporary staffing.

Healthcare provider staffing in the telehealth industry is static. Current telehealth staffing models are inefficient when there is a surplus of patients needing telehealth services and the same healthcare providers are participating on each platform. The functionality of current telehealth platforms compromises the efficiency of having every telehealth patient consult request seen by a healthcare provider in a consistent, timely manner. In addition, traditional practitioners are not trained to practice their trade virtually, and the solution is to consolidate processes and functionalities of different portals, into one platform.

The preexisting telemedicine platforms each have individual healthcare provider logins. When a provider receives a consult request from a patient in their confirmed licensed state, the provider is required to use his/her log in credentials, specific to the corresponding telemedicine platform in which the consult request originated. At the conclusion of each consultation, the provider is required to log out of the telemedicine platform in which he/she just saw the patient. With this management software, providers will have the option to auto-save log in credentials such as usernames and passwords to improve workflow and efficiency.

Most telemedicine platforms are contracted with multiple staffing agencies to help support their patient volume. Each telemedicine company has employees which manage these numerous staffing agencies to keep track of which healthcare providers are partnered with which staffing agency. Many healthcare providers belong to multiple different telemedicine platforms therefore, to see patients across all platforms, providers are required to log in and out of each individual platform to check their consult requests. As many healthcare providers utilize telehealth to supplement their full-time jobs, logging in and out of multiple platforms throughout their day can be cumbersome.

The present invention eliminates the need for physicians to check multiple different telehealth portals by consolidating each request for consultation into one organized management system. The present invention provides a seamless transition for patient requests among every telemedicine portal with whom a physician is associated.

The present invention provides a solution to telehealth staffing needs, as it provides a dynamic way to integrate provides who already belong to several telemedicine platforms, into one organized provider portal. The use of the present invention will eliminate the need for healthcare providers to log onto multiple platforms throughout the day in order to keep track of their patient requests for consultations. The present invention would improve the efficiency and productivity of each individual telemedicine portal along with every provider personal workflow. A study by one telemedicine provider found that they would be able to increase their volume of patients seen per day by at least 30% if dynamic staffing of their system was employed, to ameliorate staffing shortages.

In sum, telemedicine platforms are too dynamic to be properly staffed by traditional means, namely supplementing permanent staff with temporary staff supplied by traditional (static) temporary staffing means. The present invention discloses a dynamic temporary staffing means which is more suitable for telemedicine platforms to provide timely remote health care services, namely fulfilling telemedicine staffing short falls by supplementing permanent telemedicine platform staff members with the precisely the number of temporary staff needed to meet a staffing short fall when said short fall arises.

### SUMMARY OF THE INVENTION

The present invention uses the Internet and software configured to sequentially communicate among devices which are used by health care providers and patients to provide timely remote health care services. Said configuration incorporates multiple independent telehealth portals into one management system. From the management system/platform, healthcare providers pull patients, one at a time, from a queue of consultations deriving from the network of telehealth portals, conducting the telehealth visit with a camera, in the states they are confirmed licensed, and their specialty of practice, through the software. The present invention can provide a uniform software which enables healthcare providers to see patients from multiple different telehealth portals, improves the efficiency of the contracted telehealth portals, the productivity of the providers, as well as overall improved patient care. The present invention integrates multiple telemedicine portals into one, surmounts a significant existing challenge of telemedicine, by eliminating temporary staffing shortage suffered by telemedicine providers and thereby lessening the chances of missed patient consults, and decreases the wait time for their patients.

Present invention uses a uniform software configured to allow healthcare providers to provide telehealth services to patients from multiple different telehealth portals, while simultaneously improving the efficiency of the contracted telemedicine service provider, the productivity of the healthcare professionals, as well as overall improved patient care. The present invention employs a management software which integrates multiple telemedicine portals into one, changes the future of telemedicine, by lessening the chances of missed patient consults, and decreases the wait time for the consumer. A method for staffing a telemedicine diagnostic session and creating a receipt certifying said remote diagnostic session between a licensed medical professional and a user/patient of a telemedicine service provider is provided. Said receipt includes supporting information related to said remote diagnostic session for use in

The present invention is a system of existing devices each operated by separate entities, including: three Internet servers and two Internet enabled communication devices,

wherein said first Internet server is operated by a remote healthcare provider, for example an organization which provides the remote delivery of healthcare services, including exams and consultations, over the telecommunications infrastructure and whose services include evaluating, diagnose and treat patients without the need for an in-person visit.

wherein said first Internet enabled communication device is operated by a participating client, namely a person who is registered with said remote healthcare provider and is authorized to receive services from said remote healthcare provider.

wherein said second Internet server is operated by a telemedicine temporary staffing provider, for example an organization that finds and retains workers and offers said worker's services to said remote healthcare provider in need of short-term workers,

wherein said second Internet enabled communication device is operated by a participating medical provider, namely a medically licensed person contracted to work for said telemedicine temporary staffing provider, and

wherein said third Internet server is operated by a remote payment service, for example an organization which provides the service of making a payment made from a distance, without the payer and payee being present at the same physical location.

### BRIEF DESCRIPTION OF THE DRAWINGS

FIG. 1 shows an example flow diagram of an overview of an operation of an example telemedicine temporary staffing system, including system elements.

### DETAILED DESCRIPTION OF THE INVENTION

The embodiments of the system and variants of the system of the present invention are set forth with reference to the above drawings.

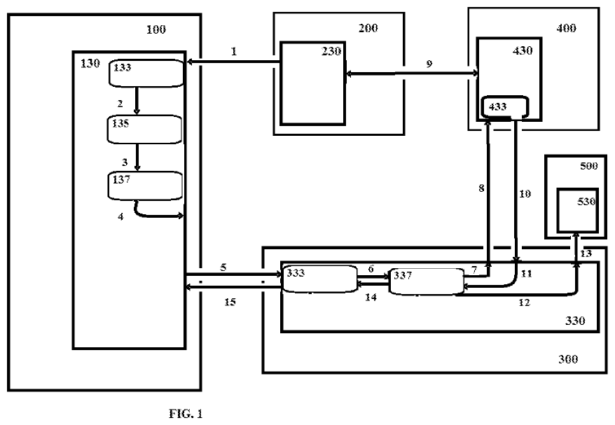

Referring now to FIG. 1, a patient having previously registered with a remote healthcare provider 100 for healthcare services by executing and sending the following said remote healthcare provider forms: summary health data, Internet contact information, service contract, Health Insurance Portability and Accountability Act privacy and data sharing consent, said patient health data, said patient becomes a participating client 200 upon said remote healthcare provider 100 receipt, acceptance and storage (in a remote healthcare provider server participating client database 137) of said patient executed forms.

Said participating client 200 initiates the use of the present invention by using an Internet enabled communication devices 230 such as phones or computers (or any other Internet communication device including but not limited to laptops, smartphones, smart gadgets, smart watches, smart and digitalized vehicles) to send a message 1 to a healthcare provider Internet server 130 which receives said message 1. Said message 1 at least contains sufficient information to identify user as a participating client and type of help requested.

Said user identity confirmation is achieved by comparing the content of said message 1 with data stored on remote healthcare provider server 130. For example, said message 1 may have a password or other content which upon being matched with data stored on remote healthcare provider server 130 will result in the confirmation of said sender of message 1 as Said participating client 200.

Similarly, the type of help requested is determined by comparing the content of said message 1 with data stored on remote healthcare provider server 130. For example, said message 1 may have a disease or other content which upon being matched with data stored on remote healthcare provider server 130 will result in the determination of the type of help requested by Said participating client 200.

As noted above, a patient may become a participating client 200 by having previously registered with said remote healthcare provider 100 for healthcare services by executing and sending the following said remote healthcare provider forms: summary health data, Internet contact information, service contract, Health Insurance Portability and Accountability Act privacy and data sharing consent. In an alternative embodiment of the present invention, said patient my become a participating client 200 by alternate means, including sending said executed forms contemporaneously with said message 1 or after sending said message 1. In yet another embodiments the information and actions associates with said executed forms may be secured by said remote healthcare provider 100 on an as needed basis.

Upon receipt of said message 1, said remote healthcare provider 100 using remote healthcare provider server 130 logs participating clients message 1 into a remote healthcare provider server log 133. After logging said message 1, said remote healthcare provider server log 133 sends a message 2 to a remote healthcare provider server queue 135 located in remote said healthcare provider Internet server 130, which is received by said remote healthcare provider server queue 135 and thereby adding said participating client 200 said message 1 assistance request to be queued among all participating clients in said remote healthcare provider server queue 135 located in said healthcare provider Internet server 130.

Said message 2 is configured contain the content of message 1 and a time of arrival. Additionally, said message 2 is formatted to allow it to be to an assistance request queue located in said remote healthcare provider server queue 135 with previously added assistance requests from all other participating clients in said remote healthcare provider server log 133 located in said remote healthcare provider Internet server queue 135.

Time waited is determined by the time which has passed since the time said message 1 was received by said healthcare provider Internet server 130. After a set amount of waiting time, wherein said set amount of waiting time is determined by remote healthcare provider 100, said remote healthcare provider server queue 135 sends message 3 including the content of said message 1 to said remote healthcare provider server participating client database 137, which is received by said remote healthcare provider server participating client database 137. Said set amount of time may vary from seconds to days.

Said set amount of waiting time may be set any time prior to, during or after message 1 is sent. In the event that no amount of time is set by said remote healthcare provider 100, then in the preferred invention, a message is displayed on said healthcare provider Internet server 130, requesting said remote healthcare provider 100 to set said set amount of time. The execution and use of the present invention will continue to wait until said set amount of waiting time is determined and entered by said remote healthcare provider 100.

Said remote healthcare provider server participating client database 137 determines the medical specialist type need to respond to assistance request found in the content of message 1 by comparing the content of message 1 and the content of the following: said remote healthcare provider consent form, said patent health data, said patient identity data including patent Internet contact information is stored in said remote healthcare provider server participating client database 137. Thus, determining the medical specialist type needed to respond to assistance request found in said message 1, is made said remote healthcare provider server participating client database 137 which has been configured to make said determination upon receipt of message 3 by said healthcare provider server participating client database,

More specifically, said remote healthcare provider server participating client database 137 is configured to incorporated and to use (upon the receipt of message 3 or any other message requesting said determination request) a compare software routine to compare the content of said message 1 and the content of the following: said remote healthcare provider consent form, said patent health data, said patient identity data including patent Internet contact information is stored in said remote healthcare provider server participating client database 137 and thereby find and associate content in said message 1 and other information associated with said participating client 200 which is associated with the need for assistance a particular type of medical type.

Upon said determination of the type of medical service said remote healthcare provider server participating client database 137 sends message 4 to said healthcare provider Internet server 130, which is received by said healthcare provider Internet server 130 requesting a telemedicine temporary staffing provider 300 to arrange for a medical specialist as determined by said remote healthcare provider server participating client database 137 to contact participating client 200. Upon receipt of message 4, healthcare provider Internet server 130 sends message 5 to said telemedicine temporary staffing provider 300, which is received by said telemedicine temporary staffing provider 300 using a telemedicine temporary staffing Internet server 330.

Said message 5 contains the content of message 1 and the content of the following: said remote healthcare provider consent form, said patent health data, said patient identity data including patent Internet contact information is stored in said remote healthcare provider server participating client database 137, as well as a request for telemedicine temporary staffing provider 300 to arrange for a medical specialist as determined by said remote healthcare provider server participating client database 137 to contact participating client 200. Said telemedicine temporary staffing provider 300 using telemedicine temporary staffing Internet server 330 logs said message 5 in a telemedicine temporary staffing Internet server log 333. Said telemedicine temporary staffing Internet server 330 sends a message 6 including the content of said messages 1-5 to said telemedicine temporary staffing Internet server participating specialist database 337, which is received by said telemedicine temporary staffing Internet server participating specialist database 337 and which is received by telemedicine temporary staffing Internet server participating specialist database.

A medical doctor having previously registered with a said telemedicine temporary staffing provider 300 for employment to provide remote healthcare services by providing an executed employment agreement and support documents including proof of all medical licenses, proof of appropriate insurance, identity data including Internet contact information is stored in said telemedicine temporary staffing Internet server participating specialist database 337. The determining the identity of a telemedicine temporary staffing provider is made by said telemedicine temporary staffing Internet server participating specialist database 337. More specifically said telemedicine temporary staffing Internet server participating specialist database 337 is configured to with and populated by said telemedicine temporary staffing provider forms executed by at least one medical doctor (said form execution is required become one of the said telemedicine temporary staffing provider 400). Said executed telemedicine temporary staffing provider forms contains said medical specialist type. The determination of the identity of the said telemedicine temporary staffing provider is made by said telemedicine temporary staffing Internet server participating specialist database 337 when it matches said medical specialist type content from message 6 with content of said telemedicine temporary staffing Internet server participating specialist database, and thereby determines which said telemedicine temporary staffing provider 400 to call which contact participating client 200.

The content said telemedicine temporary staffing provider forms executed by a medical doctor or other licensed medical professional is installed on a telemedicine temporary staffing provider identified participating medical provider device log 433 on an Internet enabled communication device 430, wherein said device 430 is a phone or computer accessible by said medical doctor, thereby enabling said medical doctor or said licensed medical professional to become a participating medical provider 400 of said telemedicine temporary staffing provider 300.

Upon the receipt of message 5 said telemedicine temporary staffing Internet server log 333 sends message 6 including the content of said message 5 to said telemedicine temporary staffing Internet server participating specialist database 337, which is received by said telemedicine temporary staffing Internet server participating specialist database 337. Said telemedicine temporary staffing Internet server participating specialist database 337 identifies said participating medical provider 400 to respond to assistance request found in the content of message 5 by comparing the content of message 5 and the content of the following: said executed employment agreement and said support documents including said proof of all medical licenses, said proof of appropriate insurance, said identity data including said Internet contact information stored in said telemedicine temporary staffing Internet server participating specialist database 337.

Upon said determination of the identity of said participating medical provider sending message 7 to said telemedicine temporary staffing Internet server 330, wherein said message 7 requests said telemedicine temporary staffing Internet server 330 to send a message 8 to identified said participating medical provider 400, which is received by said identified participating medical provider 400 using said Internet enabled communication devices 430, wherein said device 430 is a phone or computer accessible to identified said participating medical provider 400, wherein said message 8 requesting identified said participating medical provider 400 to contact participating client 200, wherein said message includes all the content of message 5, namely the content of message 1 and the content of the following: said remote healthcare provider consent form, said patent health data, said patient identity data including patent Internet contact information is stored in said remote healthcare provider server participating client database 137.

Following the receipt of said message 8 identified participating medical provider 400, said message 8 is logged on said telemedicine temporary staffing provider identified participating medical provider device log 433 and displayed on identified said participating medical provider 400.

After viewing said display identified participating medical provider 400 initiates a message exchange 9 with said participating client 200, using communication between Internet enabled communication devices 430 and an Internet enabled communication devices 230, wherein said message exchange 9 is a video and/or phone medical consultation are conducted between identified said participating medical provider 400 and said participating client 200.

Said medical consultation includes the execution of a remote diagnostic session between identified said participating medical provider with said participating client. At the beginning of said medical consultation said participating medical provider creates a receipt memorializing said session. This is done by identified said participating medical provider activating a recording device which saves a copy of all message exchange 9 communications. In the preferred embodiment of the system disclosed by the present invention, said recording device is built into said Internet enabled communication devices 430 and is automatically activated when identified participating medical provider 400 initiates said message exchange 9 with said participating client 200, using communication between Internet enabled communication devices 430 and an Internet enabled communication devices 230.

Said memorializing would be done in accordance with Health Insurance Portability and Accountability Act rules and regulations. Said memorialization will be used to update by remote healthcare provider 100 records, and as evidence of services rendered.

Upon completion of said message exchange 9, identified said participating medical provider 400 updates said telemedicine temporary staffing provider identified participating medical provider device log 433, wherein said update includes summary of content of said message exchange 9, plus any specified information requested by remote healthcare provider 100 and Said telemedicine temporary staffing provider 300, wherein said specified information is set forth in said executed employment agreement. In the preferred embodiment of the system disclosed by the present invention, said receipt memorializing said session is added to and become a part of message 10.

Upon the completion of said update, said telemedicine temporary staffing provider identified participating medical provider device log 433 sends message 10 to said telemedicine temporary staffing Internet server 330 which is received by said telemedicine temporary staffing Internet server 330.

Upon receipt of message 10 said telemedicine temporary staffing Internet server 330 sends message 11 to said telemedicine temporary staffing Internet server participating specialist database 337, which is received by said telemedicine temporary staffing Internet server participating specialist database 337 wherein said message 11 is configured to update 11 said telemedicine temporary staffing Internet server participating specialist database 337 and sends message 12 to said telemedicine temporary staffing Internet server 330, which is received by said telemedicine temporary staffing Internet server 330 to pay identified said participating medical provider 400 accordance with said executed employment agreement.

Upon receipt of message 12, said telemedicine temporary staffing Internet server 330 sends message 13 to remote payment service 500, which is received by to remote payment service 500 using remote payment service Internet server 530 to said telemedicine temporary staffing Internet server 330 to pay identified said participating medical provider 400 accordance with said executed employment agreement. After sending message 12 said telemedicine temporary staffing Internet server participating specialist database 337 and updates payment requests in said telemedicine temporary staffing Internet server participating specialist database 337, wherein said update is configured to includes the content of message 10 and message 12 and then sends message 14 to said telemedicine temporary staffing Internet server log 333, which is received by to said telemedicine temporary staffing Internet server log 333 wherein said message 14 is configured to include the content of message 10 and message 12.

Upon receipt of message 14 said telemedicine temporary staffing Internet server log 333 sends message 15 to said healthcare provider Internet server 130, which is received by said healthcare provider Internet server 130 wherein message 15 includes the content of message 11 and message 14, and an invoice requesting payment from said remote healthcare provider 100 to said telemedicine temporary staffing provider 300 for services rendered by identified said participating medical provider 400 to said participating client 200 on behalf of said remote healthcare provider 100.

In another embodiment of the present invention, message 8 will contain a list of participating clients and the type of help requested (instead of a single participating client as disclosed above) wherein each member of said list of participating clients has a drop down menu containing all the content of message 5 for each participating client on said list, namely the content of the initial contact message and the content of the following for each participating client on said list, including but not limited to remote healthcare provider consent form, patent health data, patient identity data including patent Internet contact information is stored in said remote healthcare provider server participating client database 137. After reviewing said list of participating clients, identified said participating medical provider 400, will select one participating client 200 and provide help requested in said message 1. Identified said participating medical provider 400 selects participating client 200. After said selection identified participating medical provider 400 initiates a message exchange 9 with participating client 200, using communication between Internet enabled communication devices 430 and an Internet enabled communication devices 230, wherein said message exchange 9 wherein said message exchange 9 is a video and/or phone medical consultation are conducted between identified said participating medical provider 400 and said participating client 200.

I claim:

1. A system configured to provide telemedicine temporary staffing services to a remote healthcare provider by a remote telemedicine temporary staffing provider, said services include executing remote diagnostic sessions and creating receipts memorializing said session comprising:
a remote healthcare provider Internet server operated by a remote healthcare provider,
a first Internet enabled communication device is operated by a participating client,
a telemedicine temporary staffing provider Internet server operated by a telemedicine temporary staffing provider,
a second Internet enabled communication device is operated by a participating medical provider, and
a remote payment service Internet server operated by a remote payment service,
wherein each said machine is remote from every other said machine

2. A method for operating said system disclosed in claim 1 further comprising the steps of:
a) sending a message 1 from said participating client to said remote healthcare provider using Internet communication device of said participating client,
wherein said message 1 contains a request for medical assistance,
b) receiving sending said message 1 by said remote healthcare provider from said participating client using said remote healthcare provider Internet server,
wherein said message 1 contains sufficient information to confirm sender of said message 1 as said participating client and type of help requested by said participating client; and
wherein said remote healthcare provider Internet server is configured to send message 1 to a remote healthcare provider server log,
c) logging said message 1 into said remote healthcare provider server log, upon receipt of said message 1, by said remote healthcare provider;
d) sends a message 2 from said remote healthcare provider server log to said remote healthcare provider server queue located in said remote healthcare provider Internet server,
wherein said message 2 is configured to contain the content of said message 1 and a time of arrival, and
wherein said message 2 is formatted to allow it to be to an assistance request queue located in a remote healthcare provider server queue with previously added assistance requests from all other participating clients in said remote healthcare provider server log located in said remote healthcare provider Internet server queue,
e) adding said message 2 to said remote healthcare provider server queue,
f) waiting a set amount of time,
wherein said set amount of time wherein is determined by said remote healthcare provider,
g) sending a message 3 from said healthcare provider server queue to said remote healthcare provider server participating client database,
wherein said message 3 is configured to contain the content of said message 1 and said message 2, and
wherein said message 3 is formatted to allow said message 3 to be processed by said remote healthcare provider server participating client database,
h) receiving said message 3 by said remote healthcare provider server participating client database,
wherein upon receipt of said message 3 by said remote healthcare provider server participating client database, said remote healthcare provider server participating client database initiates a determination of a medical specialist type,
i) determining said medical specialist type needed to respond to assistance request found in said message 1,
wherein said remote healthcare provider server participating client database has been populated by data for determining which said medical specialist type is best matched with which request for medical assistance,
wherein upon the completion of said determination a message 4 is initiated, and
wherein said determination said medical specialist type is made by said remote healthcare provider server participating client database which has been configured to make said determination based upon comparing the content of said message 3 and the content of said healthcare provider server participating client database,
j) sending a said message 4 from said remote healthcare provider server participating client database using said remote healthcare provider Internet server
wherein said message 4 request said remote healthcare provider Internet server requests to contact said telemedicine temporary staffing provider server using a telemedicine temporary staffing Internet server,
wherein said message 4 is sent upon the receipt of said determination,
wherein said message 4 contains the contents of said message 1, said message 2 and said message 3, and
wherein said message 4 requests said telemedicine temporary staffing provider to arrange for said medical specialist type to contact said participating client,
k) sending a message 5 from said remote healthcare provider Internet server using the Internet to said telemedicine temporary staffing provider using said telemedicine temporary staffing Internet server,
wherein said message 5 contains the content of said message 1, said message 2 and said message 3 and said message 4 and the content of said executed remote healthcare provider forms which were stored in said remote healthcare provider server participating client database, said request for said telemedicine temporary staffing provider to arrange for said medical specialist type to contact said participating client, and
wherein said message 5 is sent upon receipt of said message 4,
l) receiving said message 5 by said telemedicine temporary staffing Internet server from said remote healthcare provider Internet server by way of the Internet,
wherein said telemedicine temporary staffing Internet server is configured to send said message 5 to a telemedicine temporary staffing Internet server log, upon receipt of said message 5 by said telemedicine temporary staffing Internet server, and
wherein message 5 is formatted to be read and processed by a telemedicine temporary staffing Internet server log
m) logging said message 5 in said telemedicine temporary staffing Internet server log,
wherein said telemedicine temporary staffing Internet server log is configured to initiate sending a message 6 to said telemedicine temporary staffing Internet server participating specialist database, upon receipt of said message 5,
n) Sending said message 6 from said telemedicine temporary staffing Internet server log to said telemedicine temporary staffing Internet server participating specialist database,
wherein said message 6 contains the contents of wherein said message 1, said message 2, said message 3, said message 4 and said message 5,
o) receiving said message 6 by said telemedicine temporary staffing Internet server participating specialist database,
p) determining the identity of said telemedicine temporary staffing provider by said telemedicine temporary staffing Internet server participating specialist database,
wherein said telemedicine temporary staffing Internet server participating specialist database had been populated by said telemedicine temporary staffing provider forms executed by a medical doctor and thereby become said telemedicine temporary staffing provider information,
wherein said telemedicine temporary staffing provider forms contains said medical specialist type,
wherein said determination is made by comparing said medical specialist type content from said message 6 with content of said telemedicine temporary staffing Internet server participating specialist database,
wherein said determination is configured to initiate sending a message 7, said telemedicine temporary staffing Internet server participating,
q) sending said message 7 from telemedicine temporary staffing Internet server participating specialist database to said telemedicine temporary staffing Internet server and initiating a message 8,
r) sending said message 8 from said telemedicine temporary staffing Internet server using the Internet to identified said participating medical provider using said Internet enabled communication device,
wherein said message 8 contains the content of said message 1, said message 2, said message 3, said message 4, said message 5, said message 6 and said message 7, and
wherein said message 8 is formatted such that the content of said message 8 is logged on said telemedicine temporary staffing provider identified said participating medical provider device log located on said Internet enabled communication device, accessible to identified said participating medical provider,
s) receiving said message 8, the content of said message 8 is logged on said telemedicine temporary staffing provider identified a participating medical provider device log located on said Internet enabled communication device, accessible to identified said participating medical provider and displayed on said Internet enabled communication device, accessible to identified said participating medical provider,
t) viewing said display of said message 8 by identified said participating medical provider,
u) initiating a message exchange 9 by identified said participating medical provider with said participating client, using communication between Internet enabled communication device accessible to identified said participating medical provider and said Internet said participating client using enabled communication device assessable to said participating client,
wherein identified said participating medical provider provides medical services to said participating client,
v) executing a remote diagnostic session between identified said participating medical provider with said participating client,
w) creating a receipt memorializing said remote diagnostic session,
wherein said receipt is created by identified said participating medical provider activating a record device which saves a copy of all message exchange 9 communications,
x) completing said message exchange 9,
y) updating said telemedicine temporary staffing provider identified participating medical provider device log by identified said participating medical provider,
wherein said update includes summary of content of said message exchange 9 and a copy of said receipt memorializing said remote diagnostic session,
wherein said updating initiates a message 10 by said Internet enabled communication device available to identified said participating medical provider,
wherein said message 10 contains all information in said telemedicine temporary staffing provider identified participating medical provider device log,
wherein said message 10 contains the content of said message 8, summary of content of said message exchange 9, the content of receipt memorializing said remote diagnostic session and
wherein said message 10 is formatted to be installed in said telemedicine temporary staffing Internet server participating specialist database,
z) sending message 10 to said telemedicine temporary staffing Internet server from said Internet enabled communication device available to identified said participating medical provider,
wherein said message 10 is configured to initiate a message 11,
wherein said message 11 contains the content of said message 10,
aa) sending said message 11 from said telemedicine temporary staffing Internet server to said telemedicine temporary staffing Internet server participating specialist database,
wherein said message 11 is configured to initiate the installation of said message 11 into telemedicine temporary staffing Internet server participating specialist database,
bb) updating said telemedicine temporary staffing Internet server participating specialist database with said message 11,
wherein said updating initiates a message 12 to be sent by said telemedicine temporary staffing Internet server,
wherein said message 12 contains the content of said message 11,
cc) sending said message 12 from by said telemedicine temporary staffing Internet server to said telemedicine temporary staffing Internet server,
wherein said message 12 instructs said telemedicine temporary staffing Internet server to instruct said remote payment service to pay identified said participating medical provider accordance with said executed employment agreement for service render related to said message exchange 9,
wherein said message 12 is configured to initiate a message 13 upon sending,
dd) sending said message 13 to instruct said remote payment service to pay identified said participating medical provider accordance with said executed employment agreement for service render related to said message exchange 9,
wherein said message 13 is configured to initiate a message 14 upon sending,
wherein said message 14 contains the contents of said message 12,
ee) sending said message 14 from said telemedicine temporary staffing Internet server participating specialist database and updates payment requests in said telemedicine temporary staffing Internet server participating specialist database to said telemedicine temporary staffing Internet server log,
wherein said message 14 is configured to install the content of said telemedicine temporary staffing Internet server participating specialist database in said telemedicine temporary staffing Internet server log,
ff) receiving said message 14 by said telemedicine temporary staffing Internet server log sent by said telemedicine temporary staffing Internet server participating specialist database,
gg) updating said telemedicine temporary staffing Internet server log by installing said message 14;
wherein said message 14 is configured to initiate a message 15,
wherein said message 15 contains the content of said message 14;
hh) sending said message 15 to said remote healthcare provider Internet server from said telemedicine temporary staffing Internet server log,
wherein message said 15 includes the content of said message 14, and an invoice requesting payment from said remote healthcare provider to said telemedicine temporary staffing provider for services rendered by identified said participating medical provider to said participating client on behalf of said remote healthcare provider, and
wherein said invoice contains copy of said message 10 containing the content of said message 8, summary of content of said message exchange 9, the content of receipt memorializing said remote diagnostic session.
